(19) United States
(12) Patent Application Publication
(10) Pub. No.: US 2023/0273218 A1
(43) Pub. Date: Aug. 31, 2023

(54) KIT FOR QUANTITATIVE DETECTION USING FLUORESCENT MICROARRAY

(71) Applicant: HANGZHOU ZHEDA DIXUN BIOLOGICAL GENE ENGINEERING CO., LTD., Hangzhou, Zhejiang (CN)

(72) Inventors: Zhoujie WU, Hangzhou (CN); Yi LIU, Hangzhou (CN); Shandong WU, Hangzhou (CN); Huahao SHEN, Hangzhou (CN); Shanshan CHEN, Hangzhou (CN); Shaochang WU, Hangzhou (CN); Yifei WANG, Hangzhou (CN); Mingzhi ZHU, Hangzhou (CN); Meijie WANG, Hangzhou (CN); Chuhan CHEN, Hangzhou (CN)

(73) Assignee: HANGZHOU ZHEDA DIXUN BIOLOGICAL GENE ENGINEERING CO., LTD., Hangzhou, Zhejiang (CN)

(21) Appl. No.: 18/027,240

(22) PCT Filed: Jun. 10, 2021
(86) PCT No.: PCT/CN2021/099379

§ 371 Date: Mar. 20, 2023

(30) Foreign Application Priority Data
Apr. 16, 2021 (CN) 202110409320.2

(51) Int. Cl. G01N 33/68; G01N 33/545; G01N 33/577; G01N 33/58

(52) U.S. Cl. CPC G01N 33/6854; G01N 33/545; G01N 33/577; G01N 33/582; G01N 33/6869; G01N 33/6866; G01N 2800/60

(57) ABSTRACT

The present disclosure relates to a kit for quantitative detection using a fluorescent microarray, and belongs to the technical field of protein detection. The kit of the present disclosure includes a detection plate and a detection antibody coupled with fluorescent microspheres, where the detection plate is provided with a plurality of reaction chambers; the reaction chamber is provided with an opening, and an inner bottom surface of the reaction chamber is provided with a plurality of detection sites that are arranged side by side along a length direction of the reaction chamber at an interval. The kit of the present disclosure may detect allergen-specific IgE, IgG and IgA with high sensitivity, as well as rapidly and quantitatively detect an allergen-specific antibody IgE, IgG and IgA concentration in human serum or plasma, and may screen dozens of allergens at a time.

### CROSS-REFERENCE TO RELATED APPLICATION

This patent application claims the benefit and priority of Chinese Patent Application No. 202110409320.2, entitled “KIT FOR QUANTITATIVE DETECTION USING FLUORESCENT MICROARRAY” filed on Apr. 16, 2021, the disclosure of which is incorporated by reference herein in its entirety as part of the present application.

### TECHNICAL FIELD

The present disclosure relates to the technical field of protein detection, and in particular to a highly-sensitive kit for quantitatively detecting various cytokine levels or an allergen-specific immunoglobulin E (IgE), immunoglobulin G (IgG, including IgG4), and immunoglobulin A (IgA) concentration in human serum or plasma using a fluorescent microarray.

### BACKGROUND ART

The term cytokine storm (hypercytokineemia) was first proposed in 1993 as the pathogenesis of graft-versus-host disease (GVHD). The use of this term in infectious disease research began in early 2000, and it is used in reports on cytomegalovirus, hemophagocytic lymphohistiocytosis, influenza virus, severe acute respiratory syndrome (SARS) coronavirus (SARS-CoV), and the like. Cytokine storm is an important cause of acute respiratory distress syndrome (ARDS) and multiple organ failure, and its concentration is related to the severity and prognosis of the disease.

Cytokines are low-molecular-weight soluble proteins produced by immunogens, mitogens or other stimulants that induce a variety of cells. They have multiple functions such as regulating innate immunity, adaptive immunity, hematogenesis, and cell growth, and repairing damaged tissues. Cytokines can be divided into interleukin (IL), interferon (IFN), tumor necrosis factor (TNF), colony stimulating factor (CSF), chemokines and growth factors. Numerous cytokines promote or restrict each other in the body, forming an extremely complex immune regulatory network of cytokines. Specific cytokines exert their biological effects in three ways: autocrine, paracrine or endocrine, and have multiple characteristics such as pleiotropic, overlapping, antagonistic and synergistic properties. As a “double-edged sword”, cytokines, like other immune molecules, can not only play an immunomodulatory role, but also participate in the occurrence of a variety of diseases under certain conditions, and even trigger cytokine storm and cytokine storm syndrome, leading to Multiple organ damage, functional failure and death.

Cytokine storm is related to a variety of infectious and non-infectious diseases, and is a systemic inflammatory response induced by infections, drugs and other factors. Inflammation related to cytokine storm begins in local tissues and spreads throughout the body through the circulation. It is specifically manifested as increased blood flow, increased local temperature (fever), muscle pain/arthralgia, nausea, rash, malaise and other mild flu-like symptoms, and mobilizes the body's immune system to resist pathogen infection. Acute inflammation is also characterized by the release of pro-inflammatory cytokines or chemokines. The compensatory repair process begins shortly after the inflammation begins. In many cases, the compensatory repair process can completely restore tissue and organ function.

Pathogens try to disrupt the sophisticated immune regulatory system to evade the immune response in the state of infection, and have evolved a variety of evasion strategies to achieve large-scale replication. In some cases, pathogens can escape the immune response and will not induce an effective immune response; in other cases, certain pathogens can excessively stimulate the immune system, and when the local tissue structure is destroyed, dysregulated pro-inflammatory cytokines/chemokines may overflow into the circulatory system, causing a large-scale cascade of inflammation. When the storm strikes, single organ or multi-organ systemic inflammation is over-represented, such as pulmonary symptoms (hypoxemia, pulmonary edema caused by vascular leakage, and even ARDS), cardiovascular symptoms (hypotension, arrhythmia, myocardium damage, shock), blood system symptoms (continuous decrease in blood cells, coagulopathy, diffuse intravascular coagulation), acute kidney injury, and multiple organ failure, and even life can be threatened. This uncontrolled systemic inflammatory response is caused by the release of extreme inflammatory response mediators caused by excessive activation and expansion of primary immune cells.

Allergic diseases include allergic asthma, allergic rhinitis, allergic conjunctivitis, atopic dermatitis, urticaria, angioedema, severe anaphylaxis, and so on. The pathogenesis of allergic diseases is as follows: when a patient inhales or ingests an allergenic ingredient-containing substance (called allergen), B cells in the body are triggered to produce excessive IgE; the IgE, when contacting the allergen again in the body, will cross-link with the allergen and bind to the high-affinity receptor FccR1 on the surface of mast cells (MCs) and basophils, which results in the aggregation of FccR1 receptors and thus the activation of MCs and basophils; and MCs degranulate and release the inflammatory mediators stored in cytoplasmic granules during the activation process: histamine, which together with leukotrienes, immunoreactive prostaglandins, and IL4, IL5 and other cytokines and chemokines that are synthesized through the arachidonic acid pathway, triggers symptoms of anaphylaxis. IgE antibodies play a key role in the occurrence of allergic diseases, which is called IgE-mediated allergic reaction (namely, Gell-Coombs I hypersensitivity reaction (HR), or IgE-mediated immediate HR). IgE-mediated allergic diseases are characterized by a higher allergen-specific IgE (sIgE) antibody concentration in the circulating blood of a patient than that under normal conditions, and the more severe the disease, the higher the sIgE antibody concentration.

The clinical diagnosis of allergic diseases is provided in the practical guide for clinicians in America, where based on the medical history of a patient, allergen-specific IgE (sIgE) antibody concentrations are detected by pricking or blood sampling to screen pathogenic allergens (Siles R I, Hsieh F H. Allergy blood testing: A practical guide for clinicians. Am Clin J Medicine. 2011. 78: 585-592.). At present, methods for detecting allergen-specific IgE (sIgE) antibody concentrations in blood to screen pathogenic allergens include enzyme immunoassay (EIA), immunoblotting assay, colloidal gold-based lateral flow assay (LFA), protein microarray, etc. A method in line with the development trend and market requirements can automatically, rapidly, and accurately screen dozens of allergens at a time, with small sample usage. There are many screening products on the market. Under the brand of Phadia of ThermoFisher, the ImmunoCAP 250 system is a representative of EIA, ImmunoCAP Rapid is a representative of colloidal gold-based LFA, and ImmunoCAP ISAC is a representative of protein microarray, which pioneers the molecular diagnosis of allergens. The AllergyScreen from Mediwiss-analytic of Germany is a representative of immunoblotting assay (AlleisaScreen® Immunoblot for analysing specific IgE in human serum). Because the average IgE antibody concentration in human blood is about 0.005 μg/ml, which is 0.002% of the average concentration of total immunoglobulins, and the sIgE antibody concentration is even lower, in order to automatically, rapidly, and accurately screen dozens of allergens at a time with a small sample dosage and semi-quantitatively or quantitatively detect allergen-specific IgE (sIgE) antibody concentrations in a sample, a photoelectric signal amplification detector or a biochemical signal amplification system must be provided. For example, ImmunoCAP Rapid can only screen a dozen of allergens at a time, where without a reader, the sIgE antibody concentration can only be read by naked eyes, with a sensitivity only of 1.0 IU/ml (1 IU IgE=2.44 ng IgE); and 1.49 IU/ml is used to distinguish negative and positive results.

Cytokines are highly related to inflammation. The detection of cytokines can regulate inflammation as soon as possible, clinically guide the use of antibiotics, and assist in the diagnosis of viral infections. Current methods for detecting cytokines include flow cytometry and enzyme-linked immunosorbent assay. The cost of flow cytometry, especially the cost of the instrument is relatively high. Enzyme-linked immunoassay can only be tested individually and the amount of serum is relatively large. At present, most of the products for detecting allergy-specific IgE antibodies on the market only achieve qualitative detection, while quantitative detection requires large instruments, long experiment time and large sample size.

Cytokines, allergy-specific IgE, IgG (including IgG4), IgA, and the like are items with many detection indexes, and available methods currently on the market have problems such as large instrument, high detection cost, long test time, and large sample size.

### SUMMARY

The present disclosure aims to provide a quantitative detection kit using a fluorescent microarray. The kit of the present disclosure has high throughput, low reaction cost, high accuracy, and prominent repeatability, and may detect cytokines or allergen-specific antibodies IgE, IgG, and IgA (where IgG includes IgG4) with high sensitivity.

The present disclosure provides a kit for quantitative detection of cytokine or allegen-specific IgE, IgG including IgG4, and IgA using a fluorescent microarray, including a detection plate and a detection antibody coupled with fluorescent microspheres, where the detection plate is provided with a plurality of reaction chamber; the reaction chamber is provided with an opening, and an inner bottom surface of the reaction chamber is provided with a plurality of detection sites that are arranged side by side along a length direction of the reaction chamber at an interval.

In some embodiments, the detection sites of the detection plate may be fixed with test-cytokine-specific monoclonal antibodies or test allergens;

the detection sites fixed with test-cytokine-specific monoclonal antibodies or test allergens may be such that each detection site is coated with streptavidin that is coupled with biotin-labeled test-cytokine-specific monoclonal antibodies or biotin-labeled test allergens; the biotin-labeled test-cytokine-specific monoclonal antibodies each may be coupled to different detection sites, and the biotin-labeled test allergens each may be coupled to different detection sites.

In some embodiments, a material for the detection plate may include polystyrene (PS); an upper end of the reaction chamber may be provided with an opening; and the detection site may be a groove or a raised column.

In some embodiments, the detection plate may have 5-20 reaction chambers, and each reaction chamber may provide 20-50 detection sites.

In some embodiments, when the detection sites are fixed with test-cytokine-specific monoclonal antibodies and the detection antibody coupled with fluorescent microspheres may be a paired antibody of the test cytokine coupled with fluorescent microspheres; the test cytokine may be selected from the group consisting of IL-1beta, IL-2, IL-4, IL-5, IL-6, IL-8, IL-10, IL-12P70, IL-17A, TNF-α, IFN-γ and IFN-α.

In some embodiments, a preparation method of a detection plate with detection sites fixed with the test-cytokine-specific monoclonal antibodies may include the following steps:

coating each detection site of the detection plate with streptavidin to obtain a coated detection plate; labeling the test-cytokine-specific monoclonal antibody with biotin to obtain a biotin-labeled test-cytokine-specific monoclonal antibody; and respectively coupling the biotin-labeled test-cytokine-specific monoclonal antibodies to different detection sites of the detection plate to obtain the detection plate fixed with the test-cytokine-specific monoclonal antibodies.

In some embodiments, before the labeling with biotin, the method may further include mixing the test-cytokine-specific monoclonal antibody with 0.01M PBS buffer with a pH of 7.4 for di solving;

before a coupling with fluorescent microspheres, the method may further include mixing the paired antibody of the test cytokine with 0.01M PBS buffer with a pH of 7.4 containing 0.05% by mass Tween 20, 0.05% by mass Proclin-300, and 0.1% by mass BSA for dissolving.

In some embodiments, when the detection sites are fixed with test allergens, and the detection antibody coupled with microspheres may be an anti-human IgE, IgG including IgG4 or IgA antibody coupled with fluorescent microspheres; the test allergen may be selected from the group consisting of mite allergens, plant pollen allergens, mold allergens, animal dander allergens, insect allergens, plant food allergens, animal food allergens, and drug allergens.

In some embodiments, a preparation method of a detection plate with detection sites fixed with the test allergens may include the following steps:

coating each detection site of the detection plate with streptavidin to obtain a coated detection plate; labeling a test allergen with biotin to obtain a biotin-labeled test allergen; and

respectively coupling the biotin-labeled test allergens to different detection sites of the detection plate to obtain the detection plate fixed with the test allergens.

In some embodiments, before the labeling with biotin, the method may further include mixing the test allergens with 0.01M PBS buffer with a pH of 7.4 for dissolving;

before a coupling with fluorescent microspheres, the method may further include mixing the test allergen with the 0.01M PBS buffer with a pH of 7.4 containing 0.05% by mass Tween 20, 0.05% by mass Proclin-300, and 0.1% by mass BSA for dissolving.

The present disclosure further provides a method for high-throughput detection of cytokine, including:
(1) horizontally fixing and placing the detection plate fixed with the test cytokine-specific monoclonal antibody at room temperature;
(2) adding test serum or plasma into the reaction chambers of the detection plate, thoroughly mixing, and incubating at room temperature for 30 min to 60 min;
(3) rinsing the reaction chambers with a washing solution;
(4) adding the paired antibody of the test cytokine coupled with fluorescent microspheres into the reaction chambers, thoroughly mixing, and incubating at room temperature for 30 min to 60 min;
(5) rinsing the reaction chambers with a washing solution; and
(6) drying, and reading results with a reader.

The present disclosure also provides a method for high-throughput detection of allergen-specific IgE, IgG including IgG4, and IgA using the kit of the above technical schemes, including the following steps:
(1) horizontally fixing and placing the detection plate fixed with different allergens at room temperature;
(2) adding test serum or plasma into the reaction chambers of the detection plate, thoroughly mixing, and incubating at room temperature for 30 min to 60 min;
(3) rinsing the reaction chambers with a washing solution;
(4) adding the detection anti-human IgE, IgG (including IgG4) or IgA antibody coupled with fluorescent microspheres into the reaction chambers, thoroughly mixing, and incubating at room temperature for 30 min to 60 min;
(5) rinsing the reaction chambers with a washing solution; and
(6) drying, and reading results with a reader.

The present disclosure provides a kit for quantitative detection using a fluorescent microarray. The kit of present disclosure includes a detection plate and a detection antibody coupled with fluorescent microsphere. The present disclosure uses a dual signal amplification system of the fluorescent microsphere method and the biotin-streptavidin biological method to detect allergen-specific IgE (sIgE), IgG (sIgG, including IgG4 (sIgG4)), and IgA(sIgA) with high sensitivity. The embodiments may rapidly and quantitatively detect an allergen-specific IgE, IgG (IgG4), and IgA concentration in human serum or plasma, and together with the detection plate, dozens of allergens are screened at a time; the embodiments may also detect the cytokine concentration in human serum or plasma quickly and quantitatively with high sensitivity, as well as screen more than a dozen cytokines at a time. It is fast, accurate and highly sensitive and may be suitable for high-throughput detection.

### BRIEF DESCRIPTION OF DRAWINGS

FIG. 1 is a schematic structural diagram of the reaction chamber provided by the present disclosure;

FIG. 2 is a schematic structural diagram of an outer bottom surface of the reaction chamber provided by the present disclosure;

FIG. 3 is a schematic structural diagram of the detection plate provided by the present disclosure;

FIG. 4 is a schematic structural diagram of a fixing frame in the detection plate provided by the present disclosure; and

FIG. 5 is a schematic diagram of a back structure of the fixing frame in the detection plate provided by the present disclosure.

### DETAILED DESCRIPTION

The present disclosure provides a kit for quantitative detection of cytokine or allegen-specific IgE, IgG including IgG4, and IgA using a fluorescent microarray, including a detection plate and a detection antibody coupled with fluorescent microspheres, where the detection plate is provided with a plurality of reaction chambers; the reaction chamber is provided with an opening, and an inner bottom surface of the reaction chamber is provided with a plurality of detection sites that are arranged side by side along a length direction of the reaction chamber at an interval. In the kit of the present disclosure, fluorescent microspheres for amplifying a signal and a detection plate (biotin-streptavidin biological method) are used to obtain a dual signal amplification system, so the kit may simultaneously detect multiple indexes and greatly improve the detection efficiency and detection sensitivity by fluorescence quantification. In the present disclosure, the detection plate and the detection antibody coupled with fluorescent microspheres are placed separately.

In the present disclosure, a material for the detection plate may preferably include PS; an upper end of the reaction chamber may be provided with an opening, and an inner bottom surface of the reaction chamber is provided with a plurality of detection sites that are arranged side by side along a length direction of the reaction chamber at an interval; and the detection site may preferably be a groove or a raised column.

In the present disclosure, the reaction chamber may preferably be made of a transparent material.

In the present disclosure, an outer bottom surface of the reaction chamber is recessed inwardly at least at locations corresponding to the plurality of detection sites.

In the present disclosure, a first handle and a second handle may preferably be fixedly disposed on side walls of two ends of the reaction chamber along the length direction, respectively, and the first handle and the second handle may be embedded in a fixing frame to fix the reaction chamber on the fixing frame, thus enabling the detection plate of the present disclosure. In the present disclosure, the fixing frame may preferably be a chamber body with an open upper end, and the plurality of reaction chambers may be fixedly arranged side by side in the chamber body along a width direction of the fixing frame.

In the present disclosure, the first handle may preferably have a shape different from that of the second handle.

In the present disclosure, a first handle and a second handle may preferably be fixedly disposed on side walls of two ends of the reaction chamber along the length direction, respectively; a plurality of first handle-embedding grooves and a plurality of second handle-embedding grooves are disposed on upper end surfaces of two opposite side walls of the fixing frame along a width direction of the reaction chamber; a shape of the first handle-embedding groove matches a shape of the first handle, and a shape of the second handle-embedding groove matches a shape of the second handle; and the reaction chamber is fixedly connected to the fixing frame by embedding the first handle and the second handle into the first handle-embedding groove and the second handle-embedding groove, respectively. In the present disclosure, a plurality of weight-reducing holes may preferably be disposed on a lower bottom surface of the chamber body, and a weight-reducing slot may preferably be disposed on a lower end surface of a side wall of the fixing frame.

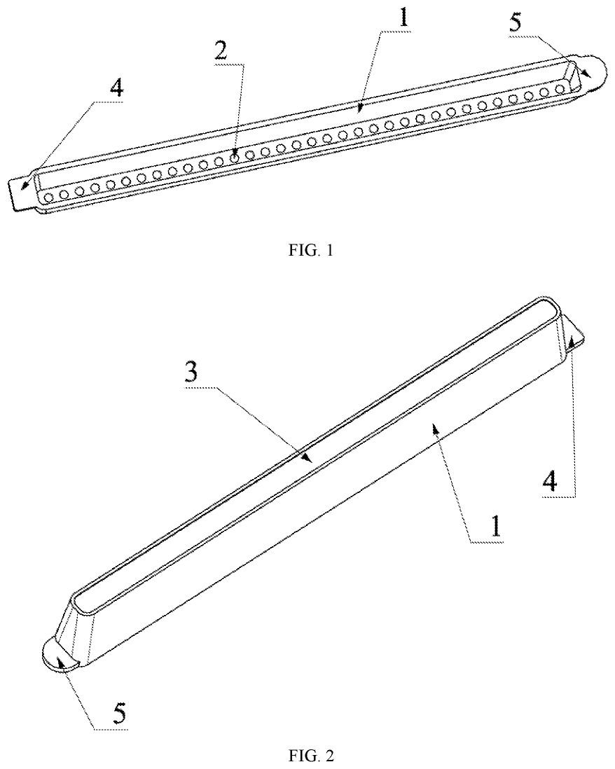

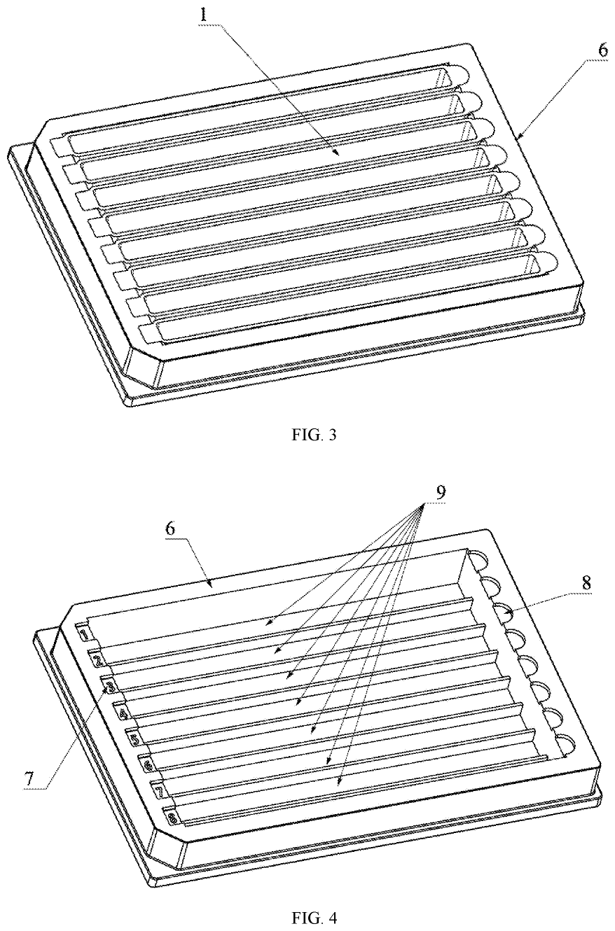

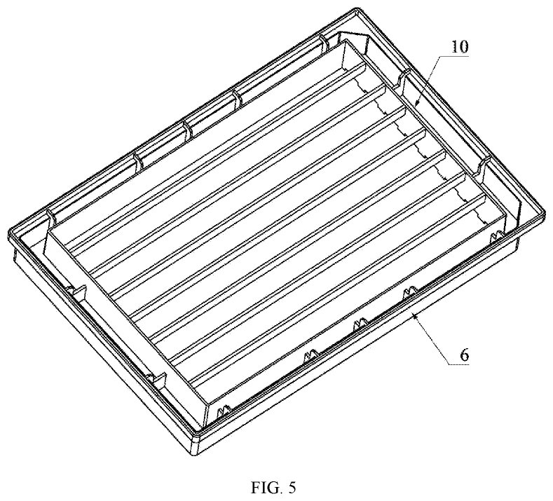

In the present disclosure, a structure of the detection plate may preferably be shown in FIG. 1 to FIG. 5, where FIG. 1 is a schematic structural diagram of the reaction chamber; FIG. 2 is a schematic structural diagram of the outer bottom surface of the reaction chamber; FIG. 3 is a schematic structural diagram of the detection plate; FIG. 4 is a schematic structural diagram of the fixing frame in the detection plate; and FIG. 5 is a schematic diagram of a back structure of the fixing frame in the detection plate. In the figures: 1 represents a reaction chamber, 2 represents a reaction site, 3 represents an anti-skidding groove, 4 represents a first handle, 5 represents a second handle, 6 represents a fixing frame, 7 represents a first handle-embedding groove, 8 represents a second handle-embedding groove, 9 represents a weight-reducing hole, and 10 represents a weight-reducing slot.

In the present disclosure, an upper end of the reaction chamber is open; an inner bottom surface of the reaction chamber is provided with a plurality of detection sites that are arranged side by side along a length direction of the reaction chamber at an interval; and the detection sites can carry proteins or antibodies, and the proteins or antibodies can be adsorbed on the detection sites. There are no special limitations on the spacing among the plurality of detection sites in the present disclosure.

In the present disclosure, the detection plate may have 5-20 reaction chambers, and each reaction chamber may preferably provide 20-50 detection sites

In the present disclosure, the kit may preferably further include a diluent and a washing solution. In the present disclosure, the diluent may preferably be a PBS buffer. In the present disclosure, the washing solution may preferably be 0.01 M, pH 7.4 PBS with 0.05% (mass percentage content) of Tween 20.

In the present disclosure, an analyte of the kit is selected from the group consisting of cytokines or allergen-specific IgE, IgG and IgA, in which IgG includes IgG4.

In the present disclosure, when an analyte of the kit is a cytokine, the detection sites of the detection plate may be fixed with test-cytokine-specific monoclonal antibodies and the detection antibody coupled with fluorescent microspheres may be a paired antibody of the test cytokine coupled with fluorescent microspheres; the test cytokine may be selected from the group consisting of IL-1beta, IL-2, IL-4, IL-5, IL-6, IL-8, IL-10, IL-12P70, IL-17A, TNF-α, IFN-γ and IFN-α.

In the present disclosure, there are no specific limitations on the source of the specific monoclonal antibody, a commercial product well known to those skilled in the art can be used, such as Anti-IL-1 beta Antibody (SinoBiological, 10139-MM07); IL-2 (abcam, ab222639); Anti-IL-4 Antibody (SinoBiological, 11846-MM04); Anti-IL-5 Antibody (SinoBiological, 15673-R001); Anti-IL-6 Antibody (SinoBiological, 10395-MM14); Anti-IL-8 Antibody (SinoBiological, 10098-MM05); Anti-IL-10 Antibody (SinoBiological, 10947-MM19); Anti-IL-12p70 Antibody (SinoBiological, CT011-R001); Anti-IL17 Antibody (SinoBiological, 12047-MM25); Anti-TNF-α Antibody (SinoBiological, 10602-MM01); Anti-IFN-γ Antibody (SinoBiological, 11725-R209); and Anti IFN-α Antibody (ProSpec-Tany, ANT-208).

In the present disclosure, there are no specific limitations on the source of the paired antibody neither, a commercial product well known to those skilled in the art can be used, such as Anti-IL-1 beta Antibody (SinoBiological, 10139-MM097); IL-2(abcam, ab222640); Anti-IL-4 Antibody (SinoBiological, 11846-MM05); Anti-IL-5 Antibody (SinoBiological, 15673-R013); Anti-IL-6 Antibody (SinoBiological, 10395-MM72); Anti-IL-8 Antibody (SinoBiological, 10098-MM18); Anti-IL-10 Antibody (SinoBiological, 10947-T16); Anti-IL-12p70 Antibody (SinoBiological, CT011-R070); Anti-IL17 Antibody (SinoBiological, 12047-MM31); TNF alpha (SinoBiological, 10602-MM08); IFN-γ (SinoBiological, 11725-R238); and Anti IFN-α Antibody (ProSpec-Tany, ANT-208).

In the present disclosure, a preparation method of a detection plate with detection sites fixed with the test-cytokine-specific monoclonal antibodies may include the following steps:
coating each detection site of the detection plate with streptavidin to obtain a coated detection plate; labeling the test-cytokine-specific monoclonal antibody with biotin to obtain a biotin-labeled test-cytokine-specific monoclonal antibody; and respectively coupling the biotin-labeled test-cytokine-specific monoclonal antibodies to different detection site of the detection plate to obtain the detection plate fixed with the test-cytokine-specific monoclonal antibodies.

In the present disclosure, streptavidin is used to coat each detection site of the plate to obtain the coated plate. there are no specific limitations on the method of coating, a conventional method for coating streptavidin well known to those skilled in the art can be adopted.

In the present disclosure, the test-cytokine-specific monoclonal antibody is labeled with biotin to obtain the biotin-labeled test-cytokine-specific monoclonal antibody. In the present disclosure, before the biotin is labeled, the test-cytokine-specific monoclonal antibody may be dissolved in 0.01M PBS buffer (pH 7.4). In the present disclosure, a volume ratio of the test-cytokine-specific monoclonal antibodies to the 0.01M PBS buffer (pH 7.4) may preferably be 1: (1-10), more preferably 1:10, to achieve an optimal sensitivity and specificity.

In the present disclosure, the biotin-labeled test-cytokine-specific monoclonal antibodies are coupled to different detection sites of the detection plate to obtain the detection plate fixed with the test-cytokine-specific monoclonal antibodies. In other words, different test-cytokine-specific monoclonal antibodies need to be fixed at different sites of the detection plate in the reaction chamber. In the present disclosure, the paired antibody of the test cytokine, before being coupled with the fluorescent microspheres, may be dissolved in 0.01M PBS buffer (pH 7.4) containing 0.05% by mass Tween 20, 0.05% by mass Proclin-300, and 0.1% by mass BSA to achieve an optimal sensitivity and specificity. In the present disclosure, a volume ratio of the paired antibody of the test cytokine to the 0.01M PBS buffer (pH 7.4) containing 0.05% by mass Tween 20, 0.05% by mass Proclin-300, and 0.1% by mass BSA may preferably be 1: (10-1000), more preferably 1:100, to achieve an optimal sensitivity and specificity. In the present disclosure, the fluorescent microsphere is purchased from invitrogen, with an Item F8807.

In the present disclosure, a method for high-throughput detection of cytokine using the kit may preferably include the following steps:
(1) horizontally fixing and placing the detection plate fixed with the test cytokine-specific monoclonal antibody at room temperature;
(2) adding test serum or plasma into the reaction chambers of the detection plate, thoroughly mixing, and incubating at room temperature for 30 min to 60 min;
(3) rinsing the reaction chambers with a washing solution;
(4) adding the paired antibody of the test cytokine coupled with fluorescent microspheres into the reaction chambers, thoroughly mixing, and incubating at room temperature for 30 min to 60 min;
(5) rinsing the reaction chambers with a washing solution; and
(6) drying, and reading results with a reader.

In the present disclosure, the detection plate fixed with different allergens is horizontally fixed and placed at room temperature (18° C. to 26° C.). In the present disclosure, the detection plate fixed with the test cytokine-specific monoclonal antibody may preferably be horizontally fixed on a plate fixing holder.

In the present disclosure, test serum or plasma is added into the reaction chambers of the detection plate, and a resulting mixture is thoroughly mixed and incubated at room temperature (18° C. to 26° C.) for 30 min to 60 min. In the present disclosure, the mixing may preferably be conducted with a mixer, and the mixer may preferably include a shaker. The shaker of the present disclosure may preferably be a WD-9405A decolorizing shaker purchased from Ward Biomedical Instrument Company.

In the present disclosure, after the incubation, the reaction chambers are rinsed with a washing solution. In the present disclosure, the rinsing may be conducted preferably for 10 s to 30 s each time, and may be conducted preferably 3 to 5 times. In the present disclosure, there are no special limitations on an amount of washing solution added to the reaction chamber during washing, and a conventional washing solution amount may be used, for example, the washing solution may be added to cover all detection sites but not overflow during reaction.

In the present disclosure, after the rinsing, the paired antibody of the test cytokine-specific monoclonal antibody coupled with fluorescent microspheres is added into the reaction chambers, and a resulting mixture is thoroughly mixed and incubated at room temperature (18° C. to 26° C.) for 30 min to 60 min.

In the present disclosure, after the incubation, the reaction chambers are rinsed with a washing solution. In the present disclosure, the rinsing may be conducted preferably for 10 s to 30 s each time, and may be conducted preferably 3 to 5 times. In the present disclosure, there are no special limitations on the amount of washing solution added to the reaction chamber during washing, and a conventional washing solution amount may be used, for example, the washing solution may be added to cover all detection sites but not overflow during reaction.

In the present disclosure, after the rinsing, the detection plate is dried, and results are read with a reader. In the present disclosure, the method of the drying may preferably include pat-drying with a hand on a paper towel. The reader of the present disclosure may preferably be a reader with a data processing function, which can quantitatively detect various allergen-specific IgE, IgG (including IgG4), or IgA concentrations in human serum or plasma. In the present disclosure, the reader may preferably be a fluorescence immunoassay analyzer (model: F10Pro) purchased from TIANJIN PAP-Days Instrument Tech Co., Ltd. The reader of the present disclosure can read a fluorescence value at a corresponding position, and then a concentration is calculated according to a standard curve.

The kit provided by the present disclosure may detect the cytokine concentration in human serum or plasma quickly and quantitatively with high sensitivity, as well as screen more than a dozen cytokines at a time. It is fast, accurate and highly sensitive and may be suitable for high-throughput detection

In the present disclosure, when an analyte of the kit is an allergen-specific IgE, IgG including IgG4, or IgA, the detection sites on the detection plate are fixed with test allergens; the detection antibody coupled with fluorescent microspheres is an anti-human IgE, IgG (including IgG4) or IgA antibody coupled with fluorescent microspheres; and the allergens may be selected form the group consisting of mite allergens, plant pollen allergens, mold allergens, animal dander allergens, insect allergens, plant food allergens, animal food allergens, and drug allergens. The allergens of the present disclosure may be extracted from natural raw materials according to conventional methods or may be obtained from recombinant expression by genetic engineering. In the present disclosure, the mite allergens may preferably include dust mites, storage mites, tropical mites, etc.; the plant pollen allergens may preferably be selected form the group consisting of tree pollen, grass pollen, weed pollen, etc.; the plant food allergens may preferably include fruits, vegetables, nuts, edible fungi, cereals, etc.; and the animal food allergens may preferably include meat, eggs, fish, crustaceans, milk, etc.

In the present disclosure, the detection plate with detection sites fixed with test allergens may be prepared by a method including the following steps:

coating each detection site of the detection plate with streptavidin to obtain a coated detection plate; labeling test allergens with biotin to obtain biotin-labeled test allergens; and

coupling the biotin-labeled test allergens to different detection sites of the detection plate to obtain the detection plate fixed with the test allergens.

In the present disclosure, each detection site of the detection plate is coated with streptavidin to obtain a coated detection plate. There are no special limitations on the method of the coating in the present disclosure, and a conventional streptavidin coating method well known to those skilled in the art may be used.

In the present disclosure, test allergens are labeled with biotin to obtain biotin-labeled test allergens. In the present disclosure, before the biotin labeling, the test allergens may preferably be dissolved in 0.1 M PBS (pH 7.4). In the present disclosure, 0.1 M PBS (pH 7.4) may preferably be used to prepare the allergens into solutions with appropriate concentrations (for example: plant pollen allergens: 0.1 mg/ml to 5.0 mg/ml; mold allergens: 1.0 mg/ml to 5.0 mg/ml; animal dander allergens: 0.5 mg/ml to 5.0 mg/ml; plant food allergens: 1.0 mg/ml to 7.0 mg/ml; animal food allergens: 1.0 mg/ml to 8.0 mg/ml; and insect allergens: 1 mg/ml to 5.0 mg/ml), thus achieving the optimal analytical performance and clinical performance for final detection.

In the present disclosure, the biotin-labeled test allergens are coupled to different detection sites of the detection plate to obtain the detection plate fixed with the test allergens. That is, in the present disclosure, different test allergens need to be fixed at different sites in the reaction chamber of the detection plate. In the present disclosure, 0.5 μL to 2 μL of a biotin-labeled test allergen may preferably be added on a detection site and react at 37° C. for 30 min to achieve fixation.

In the present disclosure, test allergens, before being coupled with fluorescent microspheres, may preferably be dissolved in 0.01 M PBS (pH 7.4) containing 0.05% of Tween 20, 0.05% of Proclin-300, and 0.1% of BSA (mass percentage content). In the present disclosure, a volume ratio of the test allergen to the 0.01 M PBS (pH 7.4) containing 0.05% of Tween 20, 0.05% of Proclin-300, and 0.1% of BSA (mass percentage content) may be preferably 1:(100-1,000) and more preferably 1:1,000. In the present disclosure, the fluorescent microspheres may preferably be purchased from abcam, with model: abcam-ab7295.

The present disclosure also provides a method for high-throughput detection of allergen-specific IgE (sIgE), IgG (sIgG, including IgG4 (sIgG4)), or IgA (sIgGA) using the kit, preferably including the following steps:
(1) horizontally fixing and placing the detection plate fixed with different allergens at room temperature;
(2) adding test serum or plasma into the reaction chambers of the detection plate, thoroughly mixing, and incubating at room temperature for 30 min to 60 min;
(3) rinsing the reaction chambers with a washing solution;
(4) adding the detection anti-human IgE, IgG (including IgG4) or IgA antibody coupled with fluorescent microspheres into the reaction chambers, thoroughly mixing, and incubating at room temperature for 30 min to 60 min;
(5) rinsing the reaction chambers with a washing solution; and
(6) drying, and reading results with a reader.

In the present disclosure, the detection plate fixed with different allergens is horizontally fixed and placed at room temperature (18° C. to 26° C.). In the present disclosure, the detection plate fixed with different allergens may preferably be horizontally fixed on a plate fixing holder.

In the present disclosure, test serum or plasma is added into the reaction chambers of the detection plate, and a resulting mixture is thoroughly mixed and incubated at room temperature (18° C. to 26° C.) for 30 min to 60 min. In the present disclosure, the mixing may preferably be conducted with a mixer, and the mixer may preferably include a shaker. The shaker of the present disclosure may preferably be a WD-9405A decolorizing shaker purchased from Ward Biomedical Instrument Company.

In the present disclosure, after the incubation, the reaction chambers are rinsed with a washing solution. In the present disclosure, the rinsing may be conducted preferably for 10 s to 30 s each time, and may be conducted preferably 3 to 5 times. In the present disclosure, there are no special limitations on an amount of washing solution added to the reaction chamber during washing, and a conventional washing solution amount may be used, for example, the washing solution may be added to cover all detection sites but not overflow during reaction.

In the present disclosure, after the rinsing, the detection anti-human IgE, IgG (including IgG4) or IgA antibody coupled with fluorescent microspheres is added into the reaction chambers, and a resulting mixture is thoroughly mixed and incubated at room temperature (18° C. to 26° C.) for 30 min to 60 min.

In the present disclosure, after the incubation, the reaction chambers are rinsed with a washing solution. In the present disclosure, the rinsing may be conducted preferably for 10 s to 30 s each time, and may be conducted preferably 3 to 5 times. In the present disclosure, there are no special limitations on the amount of washing solution added to the reaction chamber during washing, and a conventional washing solution amount may be used, for example, the washing solution may be added to cover all detection sites but not overflow during reaction.

In the present disclosure, after the rinsing, the detection plate is dried, and results are read with a reader. In the present disclosure, the method of the drying may preferably include pat-drying with a hand on a paper towel. The reader of the present disclosure may preferably be a reader with a data processing function, which can quantitatively detect various allergen-specific IgE, IgG (including IgG4), or IgA concentrations in human serum or plasma. In the present disclosure, the reader may preferably be a fluorescence immunoassay analyzer (model: F10Pro) purchased from TIANJIN PAP-Days Instrument Tech Co., Ltd. The reader of the present disclosure can read a fluorescence value at a corresponding position, and then a concentration is calculated according to a standard curve.

The kit of the present disclosure may rapidly and quantitatively detect the allergen-specific IgE (sIgE), IgG (sIgG, including IgG4 (sIgG4)) or IgA (sIgGA) concentrations in human serum or plasma with high sensitivity, as well as rapidly and accurately screen dozens of allergens at a time with high sensitivity, low cost, and portable instrument, and may be suitable for high-throughput detection.

The kit for quantitatively detecting allergens using a fluorescent microarray according to the present disclosure will be further described in detail below with reference to specific examples. The technical solutions of the present disclosure include, but are not limited to, the following examples.

### Example 1

Preparation of the Kit of the Present Disclosure

1. Fixation of cytokine-specific monoclonal antibodies on a polystyrene detection plate

A. Commercially-available streptavidin was prepared into a solution with an appropriate concentration (such as 0.1 mg/mL to 2 mg/mL) using 0.01 M PBS (pH 7.4), and 0.5 μL to 2 μL of the solution was added to each well of the reaction chambers of the detection plate to statically react overnight (more than 16 h) at 4° C.

B. The detection plate was washed with 0.5 mL to 1.5 mL of 0.01 M PBS (pH 7.4) containing 0.05% of Tween 20 once and then pat-dried.

C. 0.5 mL to 1.5 mL of 0.01 M PBS (pH 7.4) containing 2% of BSA was added to each reaction chamber of the detection plate to statically react overnight (more than 16 h) at 4° C. for blocking.

D. The detection plate was washed with 0.5 mL to 1.5 mL of 0.01 M PBS (pH 7.4) containing 0.05% of Tween 20 once and then pat-dried.

E. 0.1 mol/L PBS (pH 7.4) was used to prepare biotin-labeled allergens into solutions with appropriate concentrations (for example: IL-1beta: 0.1-7.0 mg/mL; IL-2: 2.0-5.0 mg/mL; IL-4: 0.05-3.0 mg/mL; IL-5: 1.0-8.0 mg/mL; IL-6: 1.5-10.0 mg/mL; IL-8: 0.1-3.0 mg/mL; IL-10: 2.0-5.0 mg/mL; IL-12P70: 1.5-10.0 mg/mL; IL-17A: 1.0-8.0 mg/mL; TNFa: 2.0-5.0 mg/mL; IFN-γ: 1.5-10.0 mg/mL), and 0.5 μL to 2 μL of each of the solutions was added to a well at a corresponding position and reacted at 37° C. for 30 min.

F. The detection plate was washed with 0.5 mL to 1.5 mL of 0.01 M PBS (pH 7.4) containing 0.05% of Tween 20 three times and then pat-dried for later use.

2. Coupling mixed cytokine antibodies with fluorescent microspheres

(1) IL-1beta, IL-2, IL-4, IL-5, IL-6, IL-8, IL-10, IL-12P70, IL-17A, TNF-α, IFN-γ and IFN-α were mixed in proportion (for example, 1:1:1:1:1:1:1:1:1:1:1:1), with a concentration of 1 mg/ml in PBS.

(2) Coupling with microspheres

A. 835 μL of purified water and 50 μL of a coupling buffer (a 500 mM 2-(N-morpholino) ethanesulfonic acid (MES) solution, pH 6.1) were sequentially added to a 2 mL centrifuge tube and thoroughly mixed.

B. 100 μL of 200 nm fluorescent microspheres (Invitrogen, F8807) (solid content: 2%) was added, and a resulting mixture was thoroughly mixed.

C. 50 μg of the mixed monoclonal antibody solution in step (1) was added to the centrifuge tube, and a resulting mixture was thoroughly mixed and then reacted for 30 min at room temperature on a rotation reactor (gently and continuously rotating).

D. After the reaction was completed, 10 mg/mL EDC (1-ethyl-3-(3-dimethylaminopropyl)carbodiimide hydrochloride) aqueous solution was prepared (which was prepared just before use and was used for activating carboxyl in labeling), 5 μL of the EDC solution was immediately transferred to the centrifuge tube, and a resulting mixture was thoroughly mixed by rapid pipetting.

E. Then the mixture was vortexed for thorough mixing, and then reacted for 2 h at room temperature on a rotation reactor.

F. After the reaction was completed, a resulting system was centrifuged (15,000 rpm, 8 min), and a supernatant was removed; and 1 mL of a washing buffer (0.01 M, pH 7.4 PBS with 0.05% of Tween 20) was added to the centrifuge tube, and a resulting mixture was thoroughly mixed by ultrasonic treatment (power: 15%; pulse duration: 3 s, interval: 3 s, and 1 min in total) such that a reaction product was completely dispersed.

G. Second washing of the reaction product: a resulting system was centrifuged (15,000 rpm, 10 min), and the supernatant was removed; and 1 mL of washing buffer was added, and a resulting mixture was thoroughly mixed by ultrasonic treatment (power: 10%; pulse duration: 3 s, interval: 3 s, and 1 min in total) such that the reaction product was completely dispersed.

H. 1 mL of a blocking buffer (0.01 M, pH 7.4 PBS with 0.05% of Tween 20 and 0.5% of BSA) was added to re-disperse the product by ultrasonic treatment, and a resulting reaction system reacted at room temperature for 1 h on a rotation reactor.

I. After the reaction was completed, a resulting system was centrifuged (15,000 rpm, 6 min), and the supernatant was removed; and the reaction product was washed twice with 1 mL of a storage buffer (0.01 M, pH 7.4 PBS with 0.05% of Tween 20, 0.05% of Proclin-300, and 0.1% of BSA) and finally stored in 2 mL of a storage buffer.

### Example 2

Dual signal amplification system using both the fluorescence method and the biological method (biotin-streptavidin method):

The preparation of a kit can be seen in Example 1.

The detection method was as follows:

(1) The detection plate fixed with different cytokine-specific monoclonal antibodies was horizontally placed on a special plate holder at room temperature for later use.

(2) 200 μL to 400 μL of serum or plasma was added to each reaction chamber of the detection plate, and then the plate holder was placed on a shaker and incubated at room temperature for 30 min to 60 min.

(3) The reaction chamber was rinsed 3 to 5 times using a washing solution, with 10 s to 30 s for each time.

(4) 200 μL to 400 μL of working solution of the mixed paired antibodies coupled with fluorescent microspheres was diluted with PBS in 1:(10-1,000) and then added to a reaction chamber of the detection plate, and then the detection plate was placed on a shaker and incubated at room temperature for 30 min to 60 min.

(5) The detection plate was rinsed 3 to 5 times using a washing solution, with 10 s to 30 s for each time.

(6) Then the detection plate was pat-dried, a fluorescence value at a corresponding position was read with a reader, and then a concentration was calculated according to a standard curve. Results were shown in Table 1.

### Example 3

Dual signal amplification system using the conventional ELISA double antibody sandwich method (instead of the fluorescence method and the biological method of the present disclosure)

The detection method was as follows:

(1) The cytokine monoclonal antibodies were dissolved in 0.01M PBS (pH 7.4) to form a suitable concentration (eg: IL-1beta: 0.1-7.0 mg/mL; IL-2: 2.0-5.0 mg/mL; IL-4: 0.05-3.0 mg/mL; IL-5: 1.0-8.0 mg/mL; IL-6: 1.5-10.0 mg/mL; IL-8: 0.1-3.0 mg/mL; IL-10: 2.0-5.0 mg/mL; IL-12P70: 1.5-10.0 mg/mL; IL-17A: 1.0-8.0 mg/mL; TNFa: 2.0-5.0 mg/mL; IFN-γ: 1.5-10.0 mg/mL, etc.) and 50 uL was added to the hole in the corresponding site of the microplate, and a resulting mixture was allowed to stand at 4° C. for overnight reaction (above 16 h);

(2) 150 uL/well of 0.01M, PBS (pH7.4) containing 0.05% Tween 20 was added to wash the plate once and then the plate was pat-dried.

(3) 100 uL/well of 0.01M PBS (pH7.4) containing 2% BSA was added for blocking, and a resulting mixture was allowed to stand at 4° C. for overnight reaction (above 16 h);

(4) 150 uL/well of 0.01M PBS (pH7.4) containing 0.05% Tween 20 was added to wash the ELISA plate once, then the plate was pat-dried for use.

(5) ELISA plate coated with different cytokine-specific monoclonal antibodies was placed horizontally on a shaker at room temperature for use;

(6) 20 μL to 100 μL of serum or plasma was added to the microplate, and the plate was placed on a shaker, and was incubated for 30-60 min at room temperature;

(7) Each well of the ELISA plate was washed 3 to 5 times using a washing solution, with 10 s to 30 s for each time;

(8) 20 μL to 100 μL of working solution of HRP-conjugated mixed paired antibodies (IL-1beta, IL-2, IL-4, IL-5, IL-6, IL-8, IL-10, IL-12P70, IL-17A, TNF-α, IFN-γ, and IFN-α monoclonal antibodies mixed in a certain ratio (for example: 1:1:1:1:1:1:1:1:1:1:1:1) with the same concentration as in Example 1) was diluted with PBS at a volume ratio of 1: (10˜1000), and was added to the microplate, and the microplate was placed on a mixer and was incubated at room temperature for 30 min to 60 min;

(9) the reaction chamber was rinsed 3 to 5 times using a detergent, with 10 s to 30 s for each time, and pat-dried;

(10) 20 μL to 100 μL of TMB color developing solution was added and a resulting mixture was incubated on a mixer for 5 min to 15 min at room temperature;

(11) a resulting mixture was placed on a microplate reader for interpretation.

TABLE 1
Comparison of detection results of cytokine levels in serum samples by the two systems

| Item | IL-1beta | IL-2 | IL-4 | IL-5 | IL-6 | IL-8 | IL-10 | IL-12P70 | IL-17A | TNF-a | IFN-γ | IFN-α |
|---|---|---|---|---|---|---|---|---|---|---|---|---|
| Example 2 | 60 | 100 | 10 | 10 | 6 | 10 | 50 | 5 | 10 | 100 | 10 | 12 |
| | pg/mL | pg/mL | pg/mL | pg/mL | pg/mL | pg/mL | pg/mL | pg/mL | pg/mL | pg/mL | pg/mL | pg/mL |
| Example 3 | Not | 105 | 10 | 12 | Not | 13 | 55 | Not | 11 | 112 | Not | Not |
| | detected | pg/mL | pg/mL | pg/mL | detected | pg/mL | pg/mL | detected | pg/mL | pg/mL | detected | detected |

It could be seen from the comparison results of Table 1 that the sensitivity of Example 3 was too low to detect the cytokines with low concentrations due to not taking advantage of the dual signal amplification system using both the fluorescence microspheres and biotin-streptavidin.

The results above showed that the kit of the present disclosure could provide double amplified signals and higher detection sensitivity when used for detection, and could detect multiple indexes at the same time, and the operation is simple.

### Example 4

Preparation of the Kit of the Present Disclosure

1. Fixation of allergens on a detection plate

A. Commercially-available streptavidin was prepared into a solution with an appropriate concentration (such as 0.1 mg/mL to 2 mg/mL) using 0.01 M PBS (pH 7.4), and 0.5 μL to 2 μL of the solution was added to each well of the reaction chambers of the detection plate to statically react overnight (more than 16 h) at 4° C.

B. The detection plate was washed with 0.5 mL to 1.5 mL of 0.01 M PBS (pH 7.4) containing 0.05% of Tween 20 once and then pat-dried.

C. 0.5 mL to 1.5 mL of 0.01 M PBS (pH 7.4) containing 2% of BSA was added to each reaction chamber of the detection plate to statically react overnight (more than 16 h) at 4° C. for blocking.

D. The detection plate was washed with 0.5 mL to 1.5 mL of 0.01 M PBS (pH 7.4) containing 0.05% of Tween 20 once and then pat-dried.

E. 0.1 mol/L PBS (pH 7.4) was used to prepare biotin-labeled allergens into solutions with appropriate concentrations (for example: plant pollen allergens: 0.1 mg/ml to 5.0 mg/ml; mold allergens: 1.0 mg/ml to 5.0 mg/ml; animal dander allergens: 0.5 mg/ml to 5.0 mg/ml; plant food allergens: 1.0 mg/ml to 7.0 mg/ml; animal food allergens: 1.0 mg/ml to 8.0 mg/ml; and insect allergens: 1 mg/ml to 5.0 mg/ml), and 0.5 μL to 2 μL of each of the solutions was added to a well at a corresponding position and reacted at 37° C. for 30 min.

F. The detection plate was washed with 0.5 mL to 1.5 mL of 0.01 M PBS (pH 7.4) containing 0.05% of Tween 20 three times and then pat-dried for later use.

2. Coupling anti-human IgE antibody with fluorescent microspheres

A. 835 μL of purified water and 50 μL of a coupling buffer (a 500 mM 2-(N-morpholino) ethanesulfonic acid (MES) solution, pH 6.1) were sequentially added to a 2 mL centrifuge tube and thoroughly mixed.

B. 100 μL of 100 nm to 500 nm fluorescent microspheres (Invitrogen, F8807) (solid content: 2%) was added, and a resulting mixture was thoroughly mixed.

C. 50 μg of the antibody solution was added to the centrifuge tube, and a resulting mixture was thoroughly mixed and then reacted for 30 min at room temperature on a rotation reactor (gently and continuously rotating).

D. After the reaction was completed, 5 mg/mL to 20 mg/mL EDC (1-ethyl-3-(3-dimethylaminopropyl)carbodiimide hydrochloride) aqueous solution was prepared (which was prepared just before use and was used for activating carboxyl in labeling), 1 μL to 10 μL of the EDC solution was immediately transferred to the centrifuge tube, and a resulting mixture was thoroughly mixed by rapid pipetting.

E. Then the mixture was vortexed for thorough mixing, and then reacted for 2 h at room temperature on a rotation reactor.

F. After the reaction was completed, a resulting system was centrifuged (15,000 rpm, 8 min), and a supernatant was removed; and 1 mL of a washing buffer (0.01 M, pH 7.4 PBS with 0.05% of Tween 20) was added to the centrifuge tube, and a resulting mixture was thoroughly mixed by ultrasonic treatment (power: 15%; pulse duration: 3 s, interval: 3 s, and 1 min in total) such that a reaction product was completely dispersed.

G. Second washing of the reaction product: a resulting system was centrifuged (15,000 rpm, 10 min), and the supernatant was removed; and 1 mL of washing buffer was added, and a resulting mixture was thoroughly mixed by ultrasonic treatment (power: 10%; pulse duration: 3 s, interval: 3 s, and 1 min in total) such that the reaction product was completely dispersed.

H. 1 mL of a blocking buffer (0.01 M, pH 7.4 PBS with 0.05% of Tween 20 and 0.5% of

BSA) was added to re-disperse the product by ultrasonic treatment, and a resulting reaction system reacted at room temperature for 1 h on a rotation reactor.

I. After the reaction was completed, a resulting system was centrifuged (15,000 rpm, 6 min), and the supernatant was removed; and the reaction product was washed twice with 1 mL of a storage buffer (0.01 M, pH 7.4 PBS with 0.05% of Tween 20, 0.05% of Proclin-300, and 0.1% of BSA) and finally stored in 2 mL of a storage buffer.

### Example 5

Dual signal amplification system using both the fluorescence method and the biological method (biotin-streptavidin method):

The preparation of a kit can be seen in Example 4.

The detection method was as follows:

(1) The detection plate fixed with different allergens was horizontally placed on a special plate holder at room temperature for later use.

(2) 200 μL to 400 μL of serum or plasma was added to each reaction chamber of the detection plate, and then the plate holder was placed on a shaker and incubated at room temperature for 30 min to 60 min.

(3) The reaction chamber was rinsed 3 to 5 times using a washing solution, with 10 s to 30 s for each time.

(4) 200 μL to 400 μL of the anti-human IgE antibody coupled with fluorescent microspheres was diluted with PBS in 1:(10-1,000) and then added to a reaction chamber of the detection plate, and then the detection plate was placed on a shaker and incubated at room temperature for 30 min to 60 min.

(5) The detection plate was rinsed 3 to 5 times using a washing solution, with 10 s to 30 s for each time.

(6) Then the detection plate was pat-dried, a fluorescence value at a corresponding position was read with a reader, and then a concentration was calculated according to a standard curve. Results were shown in Table 2.

### Example 6

Dual signal amplification system without the fluorescence method and the biological method:

The detection method was as follows:

(1) 0.01 M PBS (pH 7.4) was used to prepare allergens into solutions with appropriate concentrations (for example: plant pollen allergens: 0.1 mg/ml to 5.0 mg/ml; mold allergens: 1.0 mg/ml to 5.0 mg/ml; animal dander allergens: 0.5 mg/ml to 5.0 mg/ml; plant food allergens: 1.0 mg/ml to 7.0 mg/ml; animal food allergens: 1.0 mg/ml to 8.0 mg/ml; and insect allergens: 1 mg/ml to 5.0 mg/ml), and 50 μL to 100 μL of each of the solutions was added to a well at a corresponding position of an ELISA plate, and a resulting mixture was slowly mixed and reacted overnight (more than 16 h) at 4° C. on a shaker.

(2) The ELISA plate was washed with 0.01 M PBS (pH 7.4) containing 0.05% of Tween 20 (150 μL/well) once and then pat-dried.

(3) 0.01 M PBS (pH 7.4) containing 2% of BSA was added (100 μL/well), and a resulting mixture was slowly mixed and reacted overnight (more than 16 h) at 4° C. on a shaker for blocking.

(4) The ELISA plate was washed with 0.01 M PBS (pH 7.4) containing 0.05% of Tween 20 (150 μL/well) once and then pat-dried for later use.

(5) The ELISA plate coated with different allergens was horizontally placed on a plate holder at room temperature for later use.

(6) 20 μL to 100 μL of serum or plasma was added to the ELISA plate, and then the plate holder was placed on a shaker and incubated at room temperature for 30 min to 60 min.

(7) The plate was rinsed 3 to 5 times using a washing solution, with 10 s to 30 s for each time.

(8) 20 μL to 100 μL of an HRP-coupled anti-human IgE antibody working solution was added to the ELISA plate, and then the plate was placed on a shaker and incubated at room temperature for 30 min to 60 min.

(9) The plate was rinsed 3 to 5 times using a washing solution, with 10 s to 30 s for each time, and then pat-dried.

(10) 20 μL to 100 μL of a TMB chromogenic solution was added per well, and then the plate was placed on a shaker and incubated at room temperature for 5 min to 15 min.

(11) Results were read with a microplate reader.

TABLE 2
Comparison of detection results of allergen-specific IgE levels in serum samples by the two systems

| | | A | | | B | | | C | | |
|---|---|---|---|---|---|---|---|---|---|---|
| Patient | | house | house | | | Aspergillus | | Ambrosia | Artemisia | |
| | allergen | dust mite | dust | milk | Cladosporium | fumigatus | Alternaria | artemisiifolia | argyi | Apple |
| Detection | Example 5 | 4 | 2 | 2 | 1 | 1 | 2 | 3 | 2 | 1 |
| result | Example 6 | 3 | 0 | 1 | 0 | 0 | 0 | 2 | 1 | 0 |
| (level) | | | | | | | | | | |

According to detection results in the above table, it can be known that the dual signal amplification system without the fluorescence method and the biochemical method in Example 6 had a low detection sensitivity, such that sIgE antibody concentrations below grade 2 (including some at grade 2) were not detectable (semi-quantitative detection results of allergen-specific sIgE were expressed in a grading form, including grades 0 to 6 according to the international grading standard, where grade 0 indicated negative and grades 1 to 6 indicated positive; and the higher the concentration, the higher the grade).

The relationship between specific IgE concentrations and grading standards internationally was shown in Table 3:

TABLE 3
International specific IgE concentrations and grading standards

| International specific IgE | |
|---|---|
| concentration (IU/ml) | International grading standard |
| <0.35 | 0 (negative) |
| 0.35 to 0.70 | 1 (weak positive) |
| 0.71 to 3.50 | 2 (positive) |
| 3.51 to 17.50 | 3 (relatively-strong positive) |
| 17.51 to 50.0 | 4 (strong positive) |
| 50.01 to 100.0 | 5 (extra-strong positive) |
| >100.0 | 6 (extremely-strong positive) |

### Example 7

Dual signal amplification system using both the fluorescence method and the biological method (biotin-streptavidin method):

The preparation of a kit can be seen in Example 4.

The detection method was as follows:

(1) The detection plate fixed with different allergens was horizontally placed on a special plate holder at room temperature for later use.

(2) 300 μl of PBS containing 2% of BSA was added to each reaction chamber, then 20 μL to 100 μL of serum or plasma was added to each reaction chamber of the detection plate, and then the plate holder was placed on a shaker and incubated at room temperature for 30 min to 60 min.

(3) The reaction chamber was rinsed 3 to 5 times using a washing solution, with 10 s to 30 s for each time.

(4) 200 μL to 400 μL of the anti-human IgG antibody coupled with fluorescent microspheres was diluted with PBS in 1:(10-1,000) and then added to a reaction chamber of the detection plate, and then the detection plate was placed on a shaker and incubated at room temperature for 30 min to 60 min.

(5) The detection plate was rinsed 3 to 5 times using a washing solution, with 10 s to 30 s for each time.

(6) Then the detection plate was pat-dried, a fluorescence value at a corresponding position was read with a reader, and then a concentration was calculated according to a standard curve. Results were shown in Table 4.

### Example 8

Dual signal amplification system without the fluorescence method and the biological method:

The detection method was as follows:

(1) 0.01 M, pH 7.4 PBS was used to prepare allergens into solutions with appropriate concentrations (for example: plant pollen allergens: 0.1 mg/ml to 5.0 mg/ml; mold allergens: 1.0 mg/ml to 5.0 mg/ml; animal dander allergens: 0.5 mg/ml to 5.0 mg/ml; plant food allergens: 1.0 mg/ml to 7.0 mg/ml; animal food allergens: 1.0 mg/ml to 8.0 mg/ml; and insect allergens: 1 mg/ml to 5.0 mg/ml), and 50 μL to 100 μL of each of the solutions was added to a well at a corresponding position of an ELISA plate, and a resulting mixture was slowly mixed and reacted overnight (more than 16 h) at 4° C. on a shaker.

(2) The ELISA plate was washed with 0.01 M, pH 7.4 PBS with 0.05% of Tween 20 (150 μL/well) once and then pat-dried.

(3) 0.01 M PBS (pH 7.4) containing 2% of BSA was added (100 μL/well), and a resulting mixture was slowly mixed and reacted overnight (more than 16 h) at 4° C. on a shaker for blocking.

(4) The ELISA plate was washed with 0.01 M, pH 7.4 PBS with 0.05% of Tween 20 (150 μL/well) once and then pat-dried for later use.

(5) The ELISA plate coated with different allergens was horizontally placed on a plate holder at room temperature for later use.

(6) 20 μL to 100 μL of serum or plasma was added to the ELISA plate, and then the plate holder was placed on a shaker and incubated at room temperature for 30 min to 60 min.

(7) The plate was rinsed 3 to 5 times using a washing solution, with 10 s to 30 s for each time.

(8) 20 μL to 100 μL of an HRP-coupled anti-human IgG antibody working solution was added to the ELISA plate, and then the plate was placed on a shaker and incubated at room temperature for 30 min to 60 min.

(9) The plate was rinsed 3 to 5 times using a washing solution, with 10 s to 30 s for each time, and then pat-dried.

(10) 20 μL to 100 μL of a TMB chromogenic solution was added per well, and then the plate was placed on a shaker and incubated at room temperature for 5 min to 15 min.

(11) Results were read with a microplate reader.

TABLE 4
Detection results of allergen-specific IgG levels in serum samples

| Patient | | A | | | B | | | C | | |
|---|---|---|---|---|---|---|---|---|---|---|
| | allergen | egg white | milk | wheat | cod | peanut | soybean | crab | shrimp | shellfish |
| Detection | Example 7 | 2 | 1 | 2 | 1 | 3 | 2 | 4 | 2 | 1 |
| result | Example 8 | 1 | 0 | 0 | 0 | 2 | 1 | 2 | 0 | 0 |

According to the detection results in the above table, it can be known that the dual signal amplification system without the fluorescence method and the biochemical method in Example 5 had a low detection sensitivity, such that sIgG antibody concentrations below grade 2 (including some at grade 2) were not detectable.

### Example 9

Dual signal amplification system using both the fluorescence method and the biological method (biotin-streptavidin method):

The preparation of a kit can be seen in Example 4.

The detection method was as follows:

(1) The detection plate fixed with different allergens was horizontally placed on a special plate holder at room temperature for later use.

(2) 300 μl of PBS containing 2% of BSA was added to each reaction chamber, then 20 μL to 100 μL of serum or plasma was added to each reaction chamber of the detection plate, and then the plate holder was placed on a shaker and incubated at room temperature for 30 min to 60 min.

(3) The reaction chamber was rinsed 3 to 5 times using a washing solution, with 10 s to 30 s for each time.

(4) 200 μL to 400 μL of the anti-human IgG4 antibody coupled with fluorescent microspheres was diluted with PBS in 1:(10-1,000) and then added to a reaction chamber of the detection plate, and then the detection plate was placed on a shaker and incubated at room temperature for 30 min to 60 min.

(5) The detection plate was rinsed 3 to 5 times using a washing solution, with 10 s to 30 s for each time.

(6) Then the detection plate was pat-dried, a fluorescence value at a corresponding position was read with a reader, and then a concentration was calculated according to a standard curve. Results were shown in Table 5.

### Example 10

Dual signal amplification system without the fluorescence method and the biological method:

The detection method was as follows:

(1) 0.01 M, pH 7.4 PBS was used to prepare allergens into solutions with appropriate concentrations (for example: plant pollen allergens: 0.1 mg/ml to 5.0 mg/ml; mold allergens: 1.0 mg/ml to 5.0 mg/ml; animal dander allergens: 0.5 mg/ml to 5.0 mg/ml; plant food allergens: 1.0 mg/ml to 7.0 mg/ml; animal food allergens: 1.0 mg/ml to 8.0 mg/ml; and insect allergens: 1 mg/ml to 5.0 mg/ml), and 50 μL to 100 μL of each of the solutions was added to a well at a corresponding position of an ELISA plate, and a resulting mixture was slowly mixed and reacted overnight (more than 16 h) at 4° C. on a shaker.

(2) The ELISA plate was washed with 0.01 M, pH 7.4 PBS with 0.05% of Tween 20 (150 μL/well) once and then pat-dried.

(3) 0.01 M PBS (pH 7.4) containing 2% of BSA was added (100 μL/well), and a resulting mixture was slowly mixed and reacted overnight (more than 16 h) at 4° C. on a shaker for blocking.

(4) The ELISA plate was washed with 0.01 M, pH 7.4 PBS with 0.05% of Tween 20 (150 μL/well) once and then pat-dried for later use.

(5) The ELISA plate coated with different allergens was horizontally placed on a plate holder at room temperature for later use.

(6) 20 μL to 100 μL of serum or plasma was added to the ELISA plate, and then the plate holder was placed on a shaker and incubated at room temperature for 30 min to 60 min.

(7) The plate was rinsed 3 to 5 times using a washing solution, with 10 s to 30 s for each time.

(8) 20 μL to 100 μL of an HRP-coupled anti-human IgG4 antibody working solution was added to the ELISA plate, and then the plate was placed on a shaker and incubated at room temperature for 30 min to 60 min.

(9) The plate was rinsed 3 to 5 times using a washing solution, with 10 s to 30 s for each time, and then pat-dried.

(10) 20 μL to 100 μL of a TMB chromogenic solution was added per well, and then the plate was placed on a shaker and incubated at room temperature for 5 min to 15 min.

(11) Results were read with a microplate reader.

TABLE 5
Detection results of allergen-specific IgG4 levels in serum samples

| Patient | | A | | | B | | | C | | |
|---|---|---|---|---|---|---|---|---|---|---|
| | allergen | egg white | milk | wheat | cod | peanut | soybean | crab | shrimp | shellfish |
| Detection | Example 9 | 1 | 1 | 4 | 2 | 3 | 4 | 3 | 4 | 1 |
| result | Example 10 | 0 | 0 | 3 | 1 | 2 | 1 | 2 | 0 | 0 |

According to the detection results in the above table, it can be known that the dual signal amplification system without the fluorescence method and the biochemical method in Example 10 had a low detection sensitivity, such that sIgG4 antibody concentrations below grade 2 were not detectable.

### Example 11

Dual signal amplification system using both the fluorescence method and the biological method (biotin-streptavidin method):

The preparation of a kit can be seen in Example 4.

A detection method was as follows:

(1) The detection plate fixed with different allergens was horizontally placed on a special plate holder at room temperature for later use.

(2) 20 μL to 100 μL of serum or plasma was added to each reaction chamber of the detection plate, and then the plate holder was placed on a shaker and incubated at room temperature for 30 min to 60 min.

(3) The reaction chamber was rinsed 3 to 5 times using a washing solution, with 10 s to 30 s for each time.

(4) 200 μL to 400 μL of the anti-human IgA antibody coupled with fluorescent microspheres was diluted with PBS in 1:(10-1,000) and then added to a reaction chamber of the detection plate, and then the detection plate was placed on a shaker and incubated at room temperature for 30 min to 60 min.

(5) The detection plate was rinsed 3 to 5 times using a washing solution, with 10 s to 30 s for each time.

(6) Then the detection plate was pat-dried, a fluorescence value at a corresponding position was read with a reader, and then a concentration was calculated according to a standard curve. Results were shown in Table 6.

### Example 12

Dual signal amplification system without the fluorescence method and the biological method:

A detection method was as follows:

(1) 0.01 M, pH 7.4 PBS was used to prepare allergens into solutions with appropriate concentrations (for example: plant pollen allergens: 0.1 mg/ml to 5.0 mg/ml; mold allergens: 1.0 mg/ml to 5.0 mg/ml; animal dander allergens: 0.5 mg/ml to 5.0 mg/ml; plant food allergens: 1.0 mg/ml to 7.0 mg/ml; animal food allergens: 1.0 mg/ml to 8.0 mg/ml; and insect allergens: 1 mg/ml to 5.0 mg/ml), and 50 μL to 100 μL of each of the solutions was added to a well at a corresponding position of an ELISA plate, and a resulting mixture was slowly mixed and reacted overnight (more than 16 h) at 4° C. on a shaker.

(2) The ELISA plate was washed with 0.01 M, pH 7.4 PBS with 0.05% of Tween 20 (150 μL/well) once and then pat-dried.

(3) 0.01 M PBS (pH 7.4) containing 2% of BSA was added (100 μL/well), and a resulting mixture was slowly mixed and reacted overnight (more than 16 h) at 4° C. on a shaker for blocking.

(4) The ELISA plate was washed with 0.01 M, pH 7.4 PBS with 0.05% of Tween 20 (150 μL/well) once and then pat-dried for later use.

(5) The ELISA plate coated with different allergens was horizontally placed on a plate holder at room temperature for later use.

(6) 20 μL to 100 μL of serum or plasma was added to the ELISA plate, and then the plate holder was placed on a shaker and incubated at room temperature for 30 min to 60 min.

(7) The plate was rinsed 3 to 5 times using a washing solution, with 10 s to 30 s for each time.

(8) 20 μL to 100 μL of an HRP-coupled anti-human IgA antibody working solution was added to the ELISA plate, and then the plate was placed on a shaker and incubated at room temperature for 30 min to 60 min.

(9) The plate was rinsed 3 to 5 times using a washing solution, with 10 s to 30 s for each time, and then pat-dried.

(10) 20 μL to 100 μL of a TMB chromogenic solution was added per well, and then the plate was placed on a shaker and incubated at room temperature for 5 min to 15 min.

(11) Results were read with a microplate reader.

TABLE 6
Detection results of allergen-specific IgA levels in serum samples

| Patient | | A | | | B | | | C | | |
|---|---|---|---|---|---|---|---|---|---|---|
| | allergen | egg white | milk | wheat | cod | peanut | soybean | crab | shrimp | shellfish |
| Detection | Example 11 | 2 | 1 | 3 | 2 | 1 | 2 | 2 | 3 | 2 |
| result | Example 12 | 1 | 0 | 2 | 0 | 0 | 1 | 1 | 2 | 0 |

According to the detection results in the above table, it can be known that the dual signal amplification system without the fluorescence method and the biochemical method in Example 10 had a low detection sensitivity, such that sIgA antibody concentrations below grade 2 (including some at grade 2) were not detectable.

the above descriptions are merely preferred implementations of the present disclosure. It should be noted that a person of ordinary skill in art may further make several improvements and modifications without departing from the principle of the present disclosure, but such improvements and modifications should be deemed as falling within the protection scope of the present disclosure.

1-12. (canceled)

13. A kit for quantitatively detecting cytokine or allergen-specific IgE, IgG comprising IgG4, and IgA using a fluorescent microarray, the kit comprising a detection plate and a detection antibody coupled with fluorescent microspheres, wherein the detection plate is provided with a plurality of reaction chambers; the reaction chamber is provided with an opening, and an inner bottom surface of the reaction chamber is provided with a plurality of detection sites that are arranged side by side along a length direction of the reaction chamber at an interval;
an analyte comprises a cytokine or allergen-specific IgE, IgG comprising IgG4, and IgA;
a material of the detection plate comprises polystyrene (PS); an upper end of the reaction chamber is provided with an opening; an inner bottom surface of the reaction chamber is provided with a plurality of detection sites that are arranged side by side along a length direction of the reaction chamber at an interval and the detection site is a groove or a raised column;
when the analyte of the kit is a cytokine, the detection sites of the detection plate are fixed with test-cytokine-specific monoclonal antibodies and the detection antibody coupled with fluorescent microspheres is a paired antibody of the test cytokine coupled with fluorescent microspheres; the test cytokine is selected from the group consisting of IL-1beta, IL-2, IL-4, IL-5, IL-6, IL-8, IL-10, IL-12P70, IL-17A, TNF-α, IFN-γ, and IFN-α;
a preparation method of the detection plate with detection sites fixed with the test-cytokine-specific monoclonal antibodies comprises the following steps:
coating each detection site of the detection plate with streptavidin to obtain a coated detection plate; labeling the test-cytokine-specific monoclonal antibody with biotin to obtain a biotin-labeled test-cytokine-specific monoclonal antibody; and coupling the biotin-labeled test-cytokine-specific monoclonal antibodies to different detection sites of the detection plate respectively to obtain the detection plate fixed with the test-cytokine-specific monoclonal antibodies.
a preparation method of the detection plate fixed with the test-cytokine-specific monoclonal antibodies comprises the following steps:
coating each detection site of the detection plate with streptavidin to obtain a coated detection plate; labeling the test-cytokine-specific monoclonal antibody with biotin to obtain a biotin-labeled test-cytokine-specific monoclonal antibody; and coupling the biotin-labeled test-cytokine-specific monoclonal antibodies to different detection sites of the detection plate respectively to obtain the detection plate fixed with the test-cytokine-specific monoclonal antibodies.
when the analyte is allergen-specific IgE, IgG comprising IgG4, or IgA, the detection sites of the detection plate are fixed with test allergens, and the detection antibody coupled with microspheres is an anti-human IgE, IgG comprising IgG4, or IgA antibody coupled with fluorescent microspheres; the test allergen is selected from the group consisting of mite allergens, plant pollen allergens, mold allergens, animal dander allergens, insect allergens, plant food allergens, animal food allergens, and drug allergens;
a preparation method of the detection plate fixed with the test allergens comprises the following steps:
coating each detection site of the detection plate with streptavidin to obtain a coated detection plate; labeling a test allergen with biotin to obtain a biotin-labeled test allergen; and
coupling the biotin-labeled test allergens to different detection sites of the detection plate respectively to obtain the detection plate fixed with the test allergens.

14. The kit according to claim 13, wherein the detection plate has 5-20 reaction chambers and each reaction chamber provides 20-50 detection sites.

15. The kit according to claim 13, wherein the test-cytokine-specific monoclonal antibody, before being labeled with biotin, is dissolved in 0.01M PBS buffer with a pH of 7.4;
the paired antibody of the test cytokine, before being coupled with fluorescent microspheres, is dissolved in 0.01M PBS buffer with a pH of 7.4 containing 0.05% by mass Tween 20, 0.05% by mass Proclin-300, and 0.1% by mass BSA.

16. The kit according to claim 13, wherein the test-cytokine-specific allergen, before being labeled with biotin, is dissolved in 0.1M PBS buffer with a pH of 7.4;
the test allergen, before being coupled with fluorescent microspheres, is dissolved in 0.01M PBS buffer with a pH of 7.4 containing 0.05% by mass Tween 20, 0.05% by mass Proclin-300, and 0.1% by mass BSA.
